(19) United States
(12) Patent Application Publication
(10) Pub. No.: US 2025/0302449 A1
(43) Pub. Date: Oct. 2, 2025

(54) Ultrasound device capable of aligning blood vessels and probes for ultrasonic Doppler measurement

(71) Applicant: EDGECARE INC., Seoul (KR)

(72) Inventors: Il Seob SONG, Goyang-si (KR); Doo Young GO, Seoul (KR); Jung Jun KIM, Seoul (KR)

(21) Appl. No.: 19/065,127

(22) Filed: Feb. 27, 2025

(30) Foreign Application Priority Data
Apr. 1, 2024 (KR) 10-2024-0043913

(51) Int. Cl. A61B 8/00; A61B 8/08

(52) U.S. Cl. CPC A61B 8/54; A61B 8/0891; A61B 8/4477; A61B 8/4488; A61B 8/461; A61B 8/5207

(57) ABSTRACT

The ultrasound device according to the present disclosure uses the linear array to align the probe with the blood vessel, positions the probe at the center using an image, and transmits/receives scan lines (center scan lines) using two phased arrays to digitize a degree of alignment with the blood vessel, thereby more effectively aligning the blood vessel and the probe.

### BACKGROUND

### 1. Field

The present disclosure relates to an ultrasound device capable of aligning blood vessels and probes for ultrasound Doppler measurement.

### 2. Description of Related Art

When a blood vessel and an ultrasound array are not aligned, an error occurs in speed estimation using the Doppler, and as a result, a blood flow may not be accurately measured. Recently, various studies are being conducted to solve this problem.

### RELATED ART DOCUMENT

### Patent Document

Korean Patent Laid-Open Publication No. 10-2021-0032866 (published on Mar. 25, 2021)

### SUMMARY

The present disclosure provides an ultrasound device that uses the linear array to align a probe with a blood vessel, positions the probe at a center using an image, and transmits/receives scan lines (center scan lines) using two phased arrays to digitize a degree of alignment with the blood vessel, thereby more effectively aligning the blood vessel and the probe.

According to an embodiment of the present disclosure, an ultrasound device may include a linear array, a first phased array, a second phased array, and an alignment unit. The linear array may transmit a first ultrasound transmission signal and receive a first ultrasound reception signal reflected from a target object. The first phased array may be arranged in a first direction with respect to the linear array to transmit a second ultrasound transmission signal and receive a second ultrasound reception signal reflected from the target object. The second phased array may be arranged in a second direction opposite to the first direction with respect to the linear array to transmit a third ultrasound transmission signal and receive a third ultrasound reception signal reflected from the target object. The alignment unit may provide alignment information for aligning a blood vessel included in the target object and an ultrasound probe based on the first ultrasound reception signal, the second ultrasound reception signal, and the third ultrasound reception signal.

A control unit included in the ultrasound device may transmit the second ultrasound transmission signal so as to be focused in a direction perpendicular or in a predetermined angle direction with respect to a center of the second ultrasound array, and transmit the third ultrasound transmission signal so as to be focused in a direction perpendicular or at a certain angle to a center of the third ultrasound array.

The alignment unit may include a comparison unit and an information unit. The comparison unit may compare a first amplitude signal corresponding to a magnitude of the second ultrasound reception signal and a second amplitude signal corresponding to a magnitude of the third ultrasound reception signal and provide a comparison result. The information unit may provide an alignment position and direction of the ultrasound probe included in the alignment information according to the comparison result.

The comparison unit may provide the comparison result according to a similarity corresponding to a degree of similarity of the first amplitude signal and the second amplitude signal.

The comparison unit may include a first comparison unit. The first comparison unit may provide the comparison result according to a difference signal corresponding to a difference between the first amplitude signal and the second amplitude signal.

The comparison unit may include a difference calculation unit and a second comparison unit. The difference calculation unit may calculate a first difference value corresponding to a difference value between a maximum value and a minimum value of the first amplitude signal and a second difference value corresponding to a difference value between a maximum value and a minimum value of the second The second comparison unit may provide amplitude signal. the comparison result according to the first difference value and the second difference value.

The comparison unit may include an interval calculation unit and a third comparison unit. The interval calculation unit calculate may a first interval corresponding to an interval in which the magnitude of the first amplitude signal is maintained within a range of a predetermined reference magnitude based on the minimum value of the first amplitude signal and a second interval corresponding to an interval in which the magnitude of the second amplitude signal is maintained within a range of a reference magnitude based on the minimum value of the second amplitude signal. The third comparison unit may compare the first interval and the second interval and provide the comparison result.

The comparison unit may include a fourth comparison unit. The fourth comparison unit may provide the comparison result according to a multiplication signal obtained by multiplying the first amplitude signal and the second amplitude signal.

The comparison unit may include a fifth comparison unit. The fifth comparison unit may provide the comparison result according to a shape of a blood vessel included in an ultrasound image generated based on the first ultrasound reception signal.

The comparison unit may include a sixth comparison unit. The sixth comparison unit may provide the comparison result according to a difference length corresponding to a difference value between a length of a major axis and a length of a minor axis passing through a center of the blood vessel included in the ultrasound image.

In addition to the technical problems of the present disclosure described above, other features and advantages of the present disclosure will be described below, or may be clearly understood by those skilled in the art from such description and explanation.

### BRIEF DESCRIPTION OF DRAWINGS

FIG. 1 is a diagram illustrating an ultrasound device according to embodiments of the present disclosure.

FIG. 2 is a diagram for describing the ultrasound device of FIG. 1.

FIGS. 3 and 4 are diagrams for describing an operation of a control unit included in the ultrasound device of FIG. 1.

FIGS. 5 to 8 are diagrams for describing operations of a comparison unit and an information unit included in the ultrasound device of FIG. 1.

FIGS. 9 and 10 are diagrams for describing an operation of a first comparison unit included in the ultrasound device of FIG. 1.

FIGS. 11 and 12 are diagrams for describing an operation of a second comparison unit included in the ultrasound device of FIG. 1.

FIGS. 13 and 14 are diagrams for describing an operation of a third comparison unit included in the ultrasound device of FIG. 1.

FIGS. 15 and 16 are diagrams for describing operations of a fourth comparison unit, a fifth comparison unit, and a sixth comparison unit included in the ultrasound device of FIG. 1.

FIG. 17 is a diagram for describing a display unit included in the ultrasound device of FIG. 1.

FIG. 18 is a diagram illustrating an ultrasound device according to embodiments of the present disclosure.

FIG. 19 is a diagram for describing the ultrasound device of FIG. 18.

FIG. 20 is a diagram for describing a control unit included in the ultrasound device of FIG. 18.

FIG. 21 is a diagram for describing a comparison unit and an information unit included in the ultrasound device of FIG. 18.

### DETAILED DESCRIPTION

In this specification, in adding reference numerals to components throughout the drawings, it is to be noted that like reference numerals designate like components even though components are illustrated in different drawings.

On the other hand, the meaning of the terms described in the present specification should be understood as follows.

Singular expressions should be understood as including plural expressions, unless the context clearly defines otherwise, and the scope of rights should not be limited by these terms.

It should be understood that terms such as “include” and “have” do not preclude the existence or addition possibility of one or more other features or numbers, steps, operations, components, parts, or combinations thereof.

Hereinafter, preferred embodiments of the present disclosure designed to solve the above problems will be described in detail with reference to the accompanying drawings.

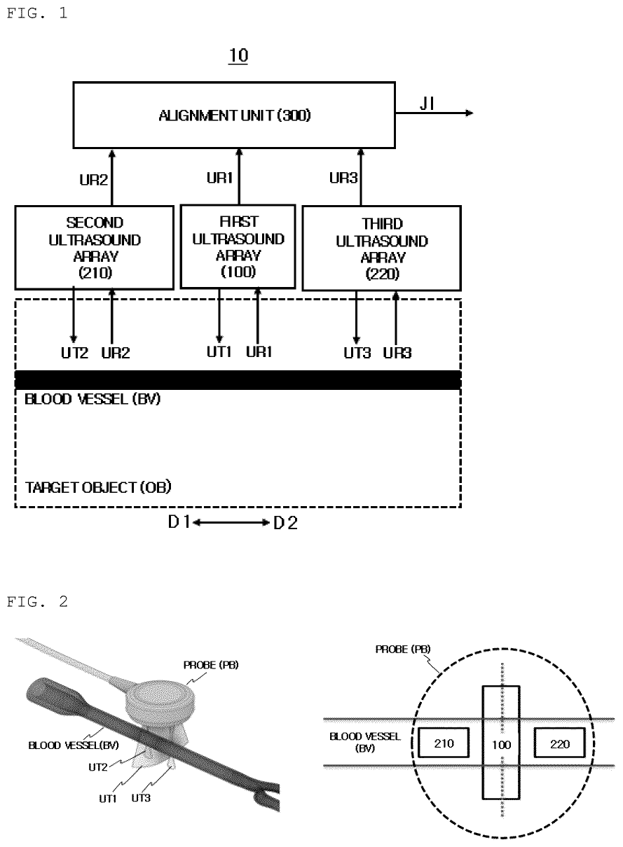

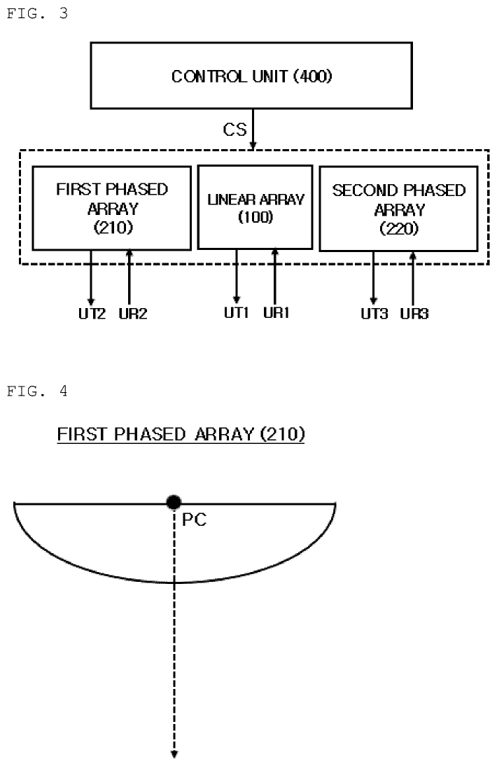

FIG. 1 is a diagram illustrating an ultrasound device according to embodiments of the present disclosure, FIG. 2 is a diagram for describing the ultrasound device of FIG. 1, and FIGS. 3 and 4 are diagrams for describing an operation of a control unit included in the ultrasound device of FIG. 1.

Referring to FIGS. 1 to 4, an ultrasound device 10 according to an embodiment of the present disclosure may include a first ultrasound array 100, a second ultrasound array 210, and a third ultrasound array 220. For example, the first ultrasound array 100 may be a linear array, the second ultrasound array 210 may be a first phased array, and the third ultrasound array 220 may be a second phased array. Here, the first ultrasound array 100 is the linear array, the second ultrasound array 210 is the first phased array, and the third ultrasound array 220 is the second phased array, and the like, but the present disclosure is not limited thereto, and each of the first ultrasound array 100 to the third ultrasound array 220 may be any one of the linear array, the first phased array, and the second phased array.

The ultrasound device 10 according to an embodiment of the present disclosure may include the linear array 100, the first phased array 210, the second phased array 220, and an alignment unit 300. The linear array 100 may transmit a first ultrasound transmission signal UT1 and receive a first ultrasound reception signal URI reflected from a target object OB. For example, an ultrasound image UI may be implemented using a first ultrasound reception signal UR1 received by the linear array 100, and an approximate location of a blood vessel BV may be identified through the ultrasound image UI. Thereafter, the linear array 100 is arranged at a location of the blood vessel BV arranged on the ultrasound image UI, and the probe PB including the linear array 100, the first phased array 210, and the second phased array 220 may be aligned with the blood vessel BV as illustrated in FIG. 2 while adjusting the first phased array 210 and the second phased array 220. Here, the location of the blood vessel BV is first identified using the linear array 100, but the present disclosure may not be limited thereto.

The first phased array 210 is arranged in a first direction D1 based on the linear array 100 to transmit the second ultrasound transmission signal UT2 and receive the second ultrasound reception signal UR2 reflected from the target object OB. For example, the first direction D1 may be the left direction based on the linear array 100, and in this case, the first phased array 210 may be arranged on the left side of the linear array 100. The first phased array 210 may transmit the second ultrasound transmission signal UT2 to be focused in a direction perpendicular to a center PC of the first phased array 210, and may receive the second ultrasound reception signal UR2 (echo signal) reflected from the target object OB. A magnitude of the second ultrasound reception signal UR2 may be large in a blood vessel BV wall arranged inside the target object OB, and may be relatively small in the blood flowing inside the blood vessel BV.

The second phased array 220 is arranged in a second direction D2 opposite to the first direction D1 with respect to the linear array 100 to transmit a third ultrasound transmission signal UT3 and receive a third ultrasound reception signal UR3 reflected from a target object OB. For example, the second direction D2 may be to the right with respect to the linear array 100, and in this case, the second phased array 220 may be arranged on a right side of the linear array 100. The second phased array 220 may transmit the third ultrasound transmission signal UT3 to be focused in a direction perpendicular to a center PC of the second phased array 220, and may receive the third ultrasound reception signal UR3 (echo signal) reflected from the target object OB. A magnitude of the third ultrasound reception signal UR3 may be large in the blood vessel BV wall arranged inside the target object OB, and may be relatively small in the blood flowing inside the blood vessel BV. FIG. 4 illustrates the transmission of the first phased array 210, which may be equally applied to the transmission of the second phased array 220.

In an embodiment, a control unit 400 included in the ultrasound device 10 may control the transmission of the second ultrasound transmission signal UT2 to be focused in a direction perpendicular or in a predetermined angle direction with respect to the center PC of the first phased array 210, and the transmission of the third ultrasound transmission signal UT3 to be focused in a direction perpendicular or at a predetermined angle direction to the center PC of the second phased array 220. For example, the control unit 400 may control the linear array 100, the first phased array 210, and the second phased array 220 through a control signal CS.

The alignment unit 300 may provide alignment information JI for aligning the target object OB or the blood vessel BV included in the target object OB and the ultrasound probe PB based on the first ultrasound reception signal UR1, the second ultrasound reception signal UR2, and the third ultrasound reception signal UR3. Here, the target object may be included inside the human body. For example, the alignment unit 300 may align the blood vessel BV and the ultrasound probe PB using the ultrasound image UI generated based on the first ultrasound reception signal UR1, and may also align the blood vessel BV and the ultrasound probe PB according to a first amplitude signal AMI and a second amplitude signal AM2 corresponding to the magnitudes of the second ultrasound: reception signal UR2 and the third ultrasound reception signal UR3.

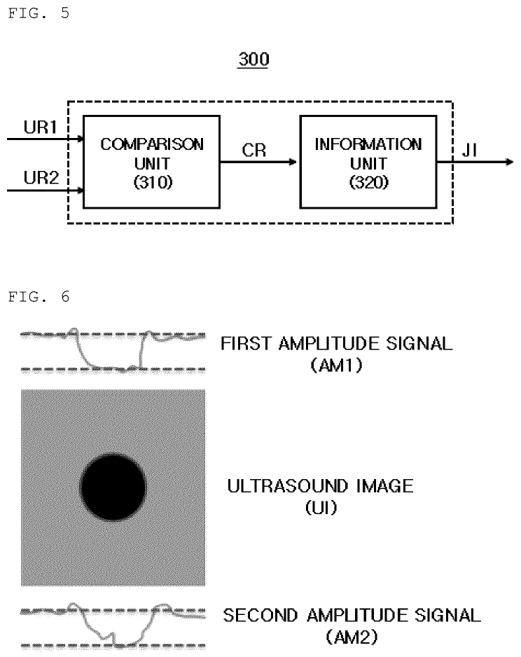

FIGS. 5 to 8 are diagrams for describing operations of a comparison unit and an information unit included in the ultrasound device of FIG. 1.

Referring to FIGS. 1 to 8, in an embodiment, the alignment unit 300 may include a comparison unit 310 and an information unit 320. The comparison unit 310 may compare a first amplitude signal AM1 corresponding to the magnitude of the second ultrasound reception signal UR2 and a second amplitude signal AM2 corresponding to the magnitude of the third ultrasound reception signal UR3 to provide a comparison result CR. FIG. 6 is a diagram illustrating the first amplitude signal AM1, the ultrasound image UI, and the second amplitude signal AM2 when the first phased array 210, the linear array 100, and the second phased array 220 are aligned in a row along the blood vessel BV as illustrated in FIG. 2.

For example, when the first phased array 210, the linear array 100, and the second phased array 220 are aligned in a row along the blood vessel BV, the first amplitude signal AM1 and the second amplitude signal AM2 may have a highest value at the location of the blood vessel BV wall and a lowest value at the location where blood is arranged. In this case, graph shapes of the first amplitude signal AM1 and the second amplitude signal AM2 may be similar.

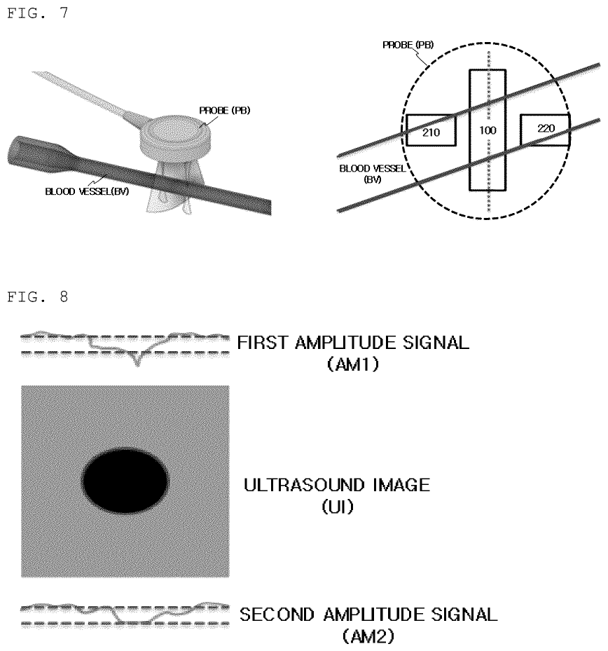

The information unit 320 may provide an alignment position and direction of the ultrasound probe PB included in the alignment information JI according to the comparison result CR. For example, as illustrated in FIG. 7, when the first phased array 210, the linear array 100, and the second phased array 220 are not aligned in a row along the blood vessel BV, the graph shapes of the first amplitude signal AM1 and the second amplitude signal AM2 may not be similar. In addition, a difference between a maximum value MX1 and a minimum value MI1 of the first amplitude signal AM1 may be small, and a difference between a maximum value MX2 and a minimum value MI2 of the second amplitude signal AM2 may be small.

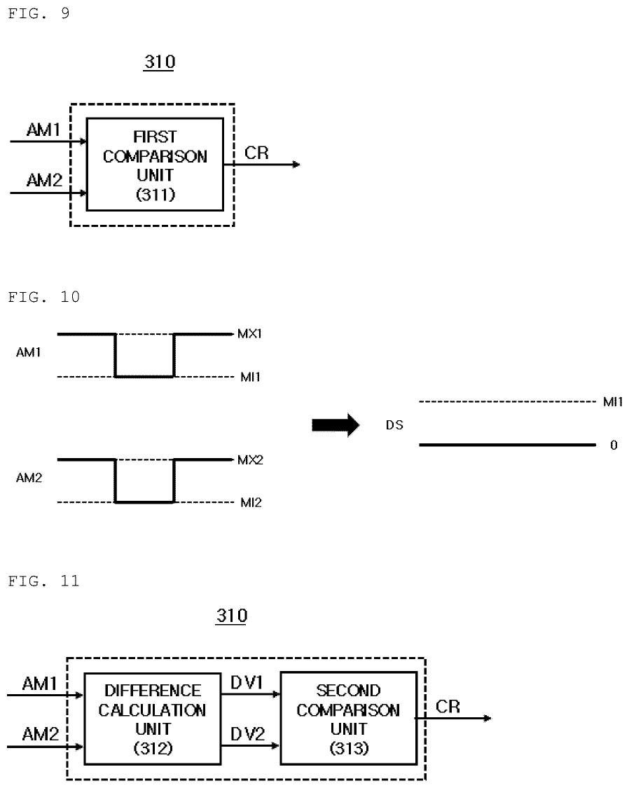

FIGS. 9 and 10 are diagrams for describing an operation of a first comparison unit included in the ultrasound device of FIG. 1.

Referring to FIGS. 1 to 10, the comparison unit 310 may provide the comparison result CR according to a similarity corresponding to the degree of similarity of the first amplitude signal AM1 and the second amplitude signal AM2. For example, the degree of similarity of the first amplitude signal AM1 and the second amplitude signal AM2 may be calculated in various ways.

In an embodiment, the comparison unit 310 may include a first comparison unit 311. The first comparison unit 311 may provide the comparison result CR according to a difference signal DS corresponding to the difference between the first amplitude signal AMI and the second amplitude signal AM2. In order to more easily describe the present disclosure, the first amplitude signal AM1 and the second amplitude signal AM2 may be represented as illustrated in FIG. 10. For example, when the difference signal DS corresponding to the difference between the first amplitude signal AM1 and the second amplitude signal AM2 is 0 as illustrated in FIG. 10 based on the location of the blood vessel BV, the first amplitude signal AM1 and the second amplitude signal AM2 may be the same. In this case, the first comparison unit 311 may determine that the first phased array 210 and the second phased array 220 are aligned in a row along the blood vessel BV. When the first comparison unit 311 determines that the first phased array 210 and the second phased array 220 are aligned in a row along the blood vessel BV, the first comparison unit 311 may provide the comparison result CR indicating that the blood vessel BV and the probe PB are aligned. Thereafter, the ultrasound device 10 according to the present disclosure may transmit and receive a Doppler signal to measure a blood flow velocity flowing in the blood vessel BV.

On the other hand, for example, when the difference signal DS corresponding to the difference between the first amplitude signal AM1 and the second amplitude signal AM2 is not 0 as illustrated in FIG. 10 but is equal to or higher than a predetermined reference level, the first comparison unit 311 may determine that the first phased array 210 and the second phased array 220 are not aligned in a row along the blood vessel BV. In this case, the comparison result CR instructing the direction or position of the first phased array 210 and the second phased array 220 to move may be provided.

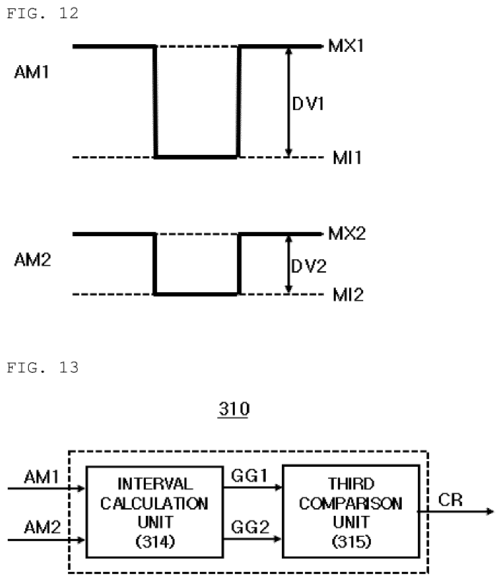

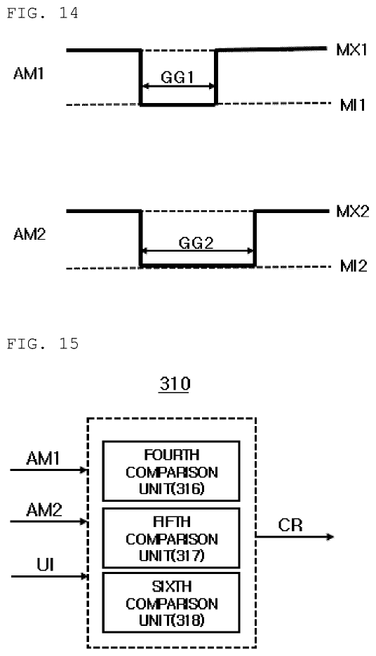

FIGS. 11 and 12 are diagrams for describing the operation of the second comparison unit included in the ultrasound device of FIG. 1, and FIGS. 13 and 14 are drawings for describing the operation of the third comparison unit included in the ultrasound device of FIG. 1.

Referring to FIGS. 1 to 14, in an embodiment, the comparison unit 310 may include a difference calculation unit 312 and a second comparison unit 313. The difference calculation unit 312 may calculate a first difference value DV1 corresponding to the difference value between the maximum value MX1 and the minimum value MI1 of the first amplitude signal AM1 and a second difference value DV2 corresponding to the difference value between the maximum value MX2 and the minimum value MI2 of the second amplitude signal AM2.

The second comparison unit 313 may provide the comparison result CR according to the first difference value DV1 and the second difference value DV2. For example, when the value obtained by subtracting the second difference value DV2 from the first difference value DV1 is greater than a predetermined reference value, the second comparison unit 313 may determine that the first phased array 210 and the second phased array 220 are not aligned in a line along the blood vessel BV. In this case, the second comparison unit 313 may provide the comparison result CR instructing the direction or position of the first phased array 210 and the second phased array 220 to move. In addition, when the value obtained by subtracting the second difference value DV2 from the first difference value DV1 is less than the predetermined reference value, the second comparison unit 313 may determine that the first phased array 210 and the second phased array 220 are aligned in a row along the blood vessel BV. In an embodiment, the comparison unit 310 may include an interval calculation unit 314 and a third comparison unit 315. The interval calculation unit 314 may calculate a first interval GG1 corresponding to an interval in which the magnitude of the first amplitude signal AM1 is maintained within a range of a predetermined reference magnitude based on the minimum value MI1 of the first amplitude signal AM1, and a second interval GG2 corresponding to an interval in which the magnitude of the second amplitude signal AM2 is maintained within a range of a reference magnitude based on the minimum value MI2 of the second amplitude signal AM2.

The third comparison unit 315 may compare the first interval GG1 and the second interval GG2 to provide the comparison result CR. For example, when the value obtained by subtracting the second interval GG2 value from the first interval GG1 value is greater than the predetermined reference interval value, the third comparison unit 315 may determine that the first phased array 210 and the second phased array 220 are not aligned in a row along the blood vessel BV. In this case, the third comparison unit 315 may provide the comparison result CR instructing the direction or position of the first phased array 210 and the second phased array 220 to move. In addition, when the value obtained by subtracting the second interval GG2 value from the first interval GG1 value is smaller than the predetermined reference interval value, the third comparison unit 315 may determine that the first phased array 210 and the second phased array 220 are aligned in a row along the blood vessel BV.

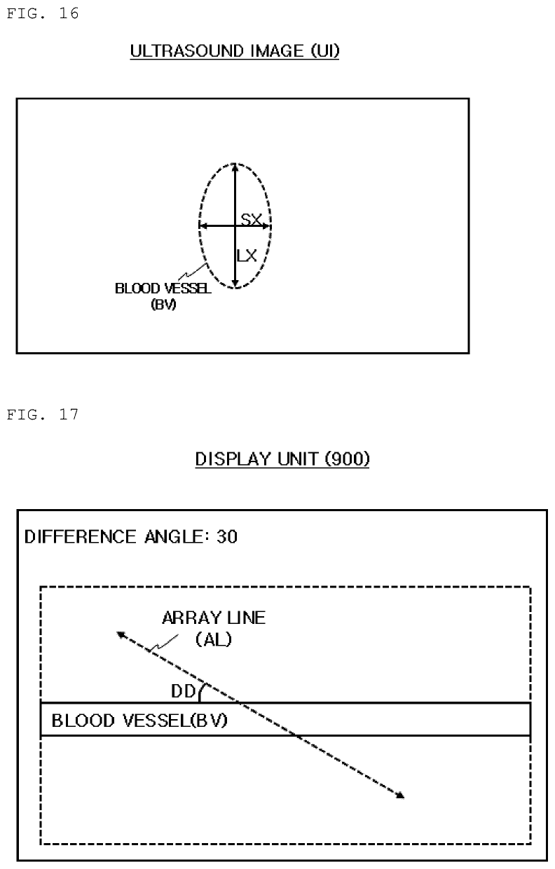

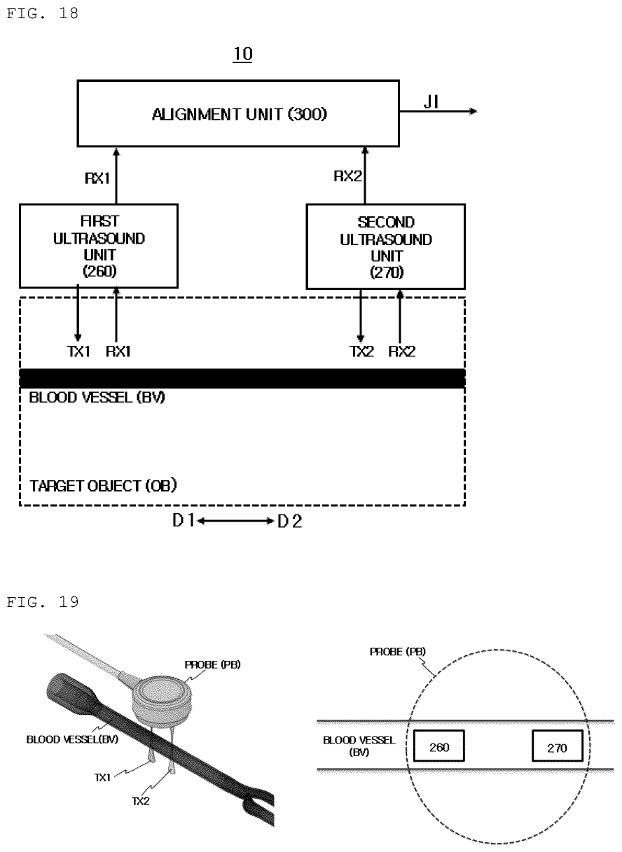

FIGS. 15 and 16 are diagrams for describing operations of a fourth comparison unit, a fifth comparison unit, and a sixth comparison unit included in the ultrasound device of FIG. 1, and FIG. 17 is a diagram for describing a display unit included in the ultrasound device of FIG. 1.

Referring to FIGS. 1 to 17, in an embodiment, the comparison unit 310 may include a fourth comparison unit 316. The fourth comparison unit 316 may provide the comparison result CR according to the multiplication signal obtained by multiplying the first amplitude signal AM1 and the second amplitude signal AM2.

In another embodiment, the comparison unit 310 may include a fifth comparison unit 317. The fifth comparison unit 317 may provide the comparison result CR according to the shape of the blood vessel BV included in the ultrasound image UI generated based on the first ultrasound reception signal UR1. For example, when the shape of the blood vessel BV included in the ultrasound image UI is not a circle, the fifth comparison unit 317 may provide the comparison result CR that the blood vessel BV and the probe PB are not arranged in a row.

In an embodiment, the comparison unit 310 may include a sixth comparison unit 318. The sixth comparison unit 318 may provide the comparison result CR according to a difference length corresponding to a difference value between a length of a major axis LX and a length of a minor axis SX passing through the center of the blood vessel BV included in the ultrasound image UI. For example, as the difference value between the length of the major axis LX and the length of the minor axis SX of the blood vessel BV included in the ultrasound image UI increases, the sixth comparison unit 318 may provide the comparison result CR indicating that the blood vessel BV and the probe PB are not aligned.

In an embodiment, the ultrasound device 10 may further include a display unit 900. The alignment information JI may include information on a difference angle DD corresponding to an angle between the blood vessel BV and the array line AL. For example, the array line AL may be a straight line connecting the center of the first phased array 210, the center of the linear array 100, and the center of the second phased array 220, and the difference angle DD corresponding to the angle between the array line AL and the blood vessel BV may be used to determine whether the blood vessel BV and the ultrasound array (the first phased array, the linear array, and the second phased array) are aligned. The display unit 900 may visually display the difference angle DD between the blood vessel BV and the array line AL, may display a numerical value of the difference angle DD, and may also display information for aligning the blood vessel and the ultrasound array in various ways. Here, the alignment information JI displayed on the display unit 900 may be in various forms including numbers, colors, symbols, etc.

In addition, in an embodiment, the ultrasound device 10 according to the present disclosure may further include an information transmission unit. The information transmission unit may transmit the alignment information JI to the outside of the ultrasound device as sound or vibration, and a user of the ultrasound device may also recognize the sound and vibration provided from the information transmission unit to confirm the alignment information JI.

The ultrasound device 10 according to the present disclosure uses the linear array 100 to align the blood vessel BV and the probe PB by positioning the probe PB at the center using the image, and transmits/receives scan lines (center scan lines) using two phased arrays to digitize the degree of alignment with the blood vessel BV, thereby more effectively align the blood vessel BV and the probe PB.

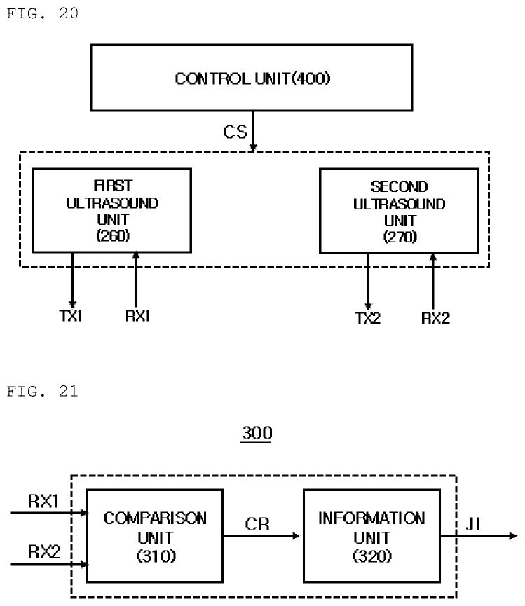

FIG. 18 is a diagram illustrating an ultrasound device according to embodiments of the present disclosure, FIG. 19 is a diagram for describing the ultrasound device of FIG. 18, FIG. 20 is a diagram for describing a control unit included in the ultrasound device of FIG. 18, and FIG. 21 is a diagram illustrating the comparison unit and the information unit included in the ultrasound device of FIG. 18

Referring to FIGS. 18 to 21, the ultrasound device 10 according to the present disclosure may include a first ultrasound unit 260, a second ultrasound unit 270, and an alignment unit 300. Here, each of the first ultrasound unit 260 and the second ultrasound unit 270 may be composed of one ultrasound element or may be composed of a plurality of ultrasound elements.

The first ultrasound unit 260 may transmit a first transmission signal TX1 corresponding to the ultrasound signal to the target object OB and receive a first reception signal RX1 reflected from the target object OB. The second ultrasound unit 270 may transmit a second transmission signal TX2 corresponding to the ultrasound signal to the target object OB and receive a second reception signal RX2 reflected from the target object OB. Here, the first ultrasound unit 260 may correspond to the second ultrasound array of FIG. 1, and the second ultrasound unit 270 may correspond to the third ultrasound array of FIG. 1. However, each of the first ultrasound unit 260 and the second ultrasound unit 270 may be different from the second ultrasound array 210 and the third ultrasound array 220 of FIG. 1 in that they may be configured with one or more elements.

In addition, the first transmission signal TX1 may correspond to the second ultrasound transmission signal UT2 of FIG. 1, and the second transmission signal TX2 may correspond to the third ultrasound transmission signal UT3 of FIG. 1. In addition, the first reception signal RX1 may correspond to the second ultrasound reception signal UR2 of FIG. 1, and the second reception signal RX2 may correspond to the third ultrasound reception signal UR3 of FIG. 1.

The alignment unit 300 may provide the alignment information JI for aligning the target object OB and the ultrasound probe PB based on the first reception signal RX1 and the second reception signal RX2. Here, the ultrasound probe PB may include the first ultrasound unit 260 and the second ultrasound unit 270. The ultrasound probe PB of FIG. 18 may have the same structure as the ultrasound probe PB of FIG. 1 except for the first ultrasound array 100. The contents of the ultrasound device 10 described in FIGS. 1 to 17 may also be applied to the ultrasound device 10 described in FIGS. 18 to 21.

In an embodiment, the control unit 400 included in the ultrasound device 10 may transmit the first transmission signal TX1 to be focused in a direction perpendicular or in a predetermined angle direction with respect to the center of the first ultrasound unit 260, and may transmit the second transmission signal TX2 to be focused in a direction perpendicular or in a predetermined angle direction with respect to the center of the second ultrasound unit 270.

In an embodiment, the alignment unit 300 may include the comparison unit 310 and the information unit 320. The comparison unit 310 may compare the first amplitude signal AM1 corresponding to the magnitude of the first reception signal RX1 and the second amplitude signal AM2 corresponding to the magnitude of the second reception signal RX2 to provide the comparison result CR. The information unit 320 may provide the alignment position and direction of the ultrasound probe PB included in the alignment information JI according to the comparison result CR.

According to the present disclosure as described above, the following effects are obtained.

The ultrasound device according to the present disclosure uses the linear array to align the probe with the blood vessel, positions the probe at the center using an image, and transmits/receives the scan lines using two phased arrays to digitize the degree of alignment with the blood vessel, thereby more effectively aligning the blood vessel and the probe.

In addition, other features and advantages of the present disclosure may be newly understood through the embodiments of the present disclosure.

What is claimed is:

1. An ultrasound device, comprising:
a first ultrasound array that transmits a first ultrasound transmission signal and receives a first ultrasound reception signal reflected from a target object;
a second ultrasound array that is arranged in a first direction based on the first ultrasound array to transmit a second ultrasound transmission signal and receive a second ultrasound reception signal reflected from the target object;
a third ultrasound array that is arranged in a second direction opposite to the first direction based on the first ultrasound array to transmit a third ultrasound transmission signal and receive a third ultrasound reception signal reflected from the target object; and
an alignment unit that provides alignment information for aligning the target object and the ultrasound probe based on the first ultrasound reception signal, the second ultrasound reception signal, and the third ultrasound reception signal.

2. The ultrasound device of claim 1, wherein a control unit included in the ultrasound device transmits the second ultrasound transmission signal so as to be focused in a direction perpendicular or in a predetermined angle direction with respect to a center of the second ultrasound array, and transmits the third ultrasound transmission signal so as to be focused in a direction perpendicular or in a predetermined angle direction with respect to a center of the third ultrasound array.

3. The ultrasound device of claim 2, wherein the alignment unit includes:
a comparison unit that compares a first amplitude signal corresponding to a magnitude of the second ultrasound reception signal and a second amplitude signal corresponding to a magnitude of the third ultrasound reception signal and provides a comparison result; and
an information unit that provides an alignment position and direction of the ultrasound probe included in the alignment information according to the comparison result.

4. The ultrasound device of claim 3, wherein the comparison unit provides the comparison result according to a similarity corresponding to a degree of similarity of the first amplitude signal and the second amplitude signal.

5. The ultrasound device of claim 4, wherein the comparison unit includes a first comparison unit that provides the comparison result according to a difference signal corresponding to a difference between the first amplitude signal and the second amplitude signal.

6. The ultrasound device of claim 5, wherein the comparison unit includes:
a difference calculation unit that calculates a first difference value corresponding to a difference value between a maximum value and a minimum value of the first amplitude signal and a second difference value corresponding to a difference value between a maximum value and a minimum value of the second amplitude signal; and
a second comparison unit that provides the comparison result according to the first difference value and the second difference value.

7. The ultrasound device of claim 6, wherein the comparison unit includes:
an interval calculation unit that calculates a first interval corresponding to an interval in which the magnitude of the first amplitude signal is maintained within a range of a predetermined reference magnitude based on the minimum value of the first amplitude signal and a second interval corresponding to an interval in which the magnitude of the second amplitude signal is maintained within a range of a reference magnitude based on the minimum value of the second amplitude signal; and
a third comparison unit that compares the first interval and the second interval and provides the comparison result.

8. The ultrasound device of claim 7, wherein the comparison unit includes a fourth comparison unit that provides the comparison result according to a multiplication signal obtained by multiplying the first amplitude signal and the second amplitude signal.

9. The ultrasound device of claim 8, wherein the comparison unit includes a fifth comparison unit that provides the comparison result according to a shape of a blood vessel included in an ultrasound image generated based on the first ultrasound reception signal.

10. The ultrasound device of claim 9, wherein the comparison unit includes a sixth comparison unit that provides the comparison result according to a difference length corresponding to a difference value between a length of a major axis and a length of a minor axis passing through a center of the blood vessel included in the ultrasound image.

11. The ultrasound device of claim 10, further comprising:
a display unit that displays the alignment information.

12. The ultrasound device of claim 10, further comprising:
an information transmission unit that transmits the alignment information as sound or vibration.

13. An ultrasound device, comprising:
a first ultrasound unit that transmits a first transmission signal corresponding to an ultrasound signal to a target object and receives a first reception signal reflected from the target object;
a second ultrasound unit that transmits a second transmission signal corresponding to the ultrasound signal to the target object and receives a second reception signal reflected from the target object; and
an alignment unit that provides alignment information for aligning the target object and an ultrasound probe based on the first reception signal and the second reception signal.

14. The ultrasound device of claim 13, wherein a control unit included in the ultrasound device transmits the first transmission signal to be focused in a direction perpendicular or in a predetermined angle direction with respect to a center of the first ultrasound unit, and
transmits the second transmission signal to be focused in a direction perpendicular or in a predetermined angle direction with respect to a center of the second ultrasound unit.

15. The ultrasound device of claim 14, wherein the alignment unit includes:
a comparison unit that compares a first amplitude signal corresponding to a magnitude of the first reception signal and a second amplitude signal corresponding to a magnitude of the second reception signal and provides a comparison result; and
an information unit that provides an alignment position and direction of the ultrasound probe included in the alignment information according to the comparison result.

16. The ultrasound device of claim 15, wherein the comparison unit provides the comparison result according to a similarity corresponding to a degree of similarity of the first amplitude signal and the second amplitude signal.
